(19) United States
(12) Patent Application Publication
(10) Pub. No.: US 2015/0030548 A1
(43) Pub. Date: Jan. 29, 2015

(54) EXTERNAL PREPARATION FOR SKIN

(71) Applicants: Zahariah Ismail, Selangor Darul Ehsan (MY); Mohd Suria Affandi Yusoff, Selangor Darul Ehsan (MY); Mamot Bin Said, Selangor Darul Ehsan (MY); Nurulhaine Bt. Ramlee, Selangor Darul Ehsan (MY)

(72) Inventors: Zahariah Ismail, Selangor Darul Ehsan (MY); Mohd Suria Affandi Yusoff, Selangor Darul Ehsan (MY); Mamot Bin Said, Selangor Darul Ehsan (MY); Nurulhaine Bt. Ramlee, Selangor Darul Ehsan (MY)

(73) Assignees: Universiti Kebangsaan Malaysia; Sime Darby Malaysia Berhad

(21) Appl. No.: 14/234,129

(22) PCT Filed: Jun. 12, 2012
(86) PCT No.: PCT/MY2012/000119

§ 371 Date: May 20, 2014

(30) Foreign Application Priority Data
Jul. 21, 2011 (MY) PI 2011003421

(51) Int. Cl. A61K 8/97; A61Q 19/02

(52) U.S. Cl. CPC A61K 8/975; A61Q 19/02
USPC 424/59; 424/62

(57) ABSTRACT

The present invention relates to an external preparation for skin containing pollen extract of palm of the genus Elaeis guineensis in an amount between 0.05% and 1.00% by weight, based on the total weight of the external preparation, for whitening skin.

### FIELD OF THE INVENTION

The present invention relates to an external preparation for skin containing pollen extract. More particularly, the present invention relates to an external preparation for skin containing pollen extract of oil palm of the genus Elaeis guineensis, for skin whitening.

### BACKGROUND

In recent years, cosmetic compositions have been developed to reduce the amount of melanin in skin and accordingly, whiten the skin. Efforts for such development have been focused on the development of whitening agents for inhibiting the activity of tyrosinase, which plays an important role in the biosynthesis of melanin.

Studies have been carried to find whitening active ingredients from natural substances. Among these ingredients, pluralities of plants extracts were found to inhibit the production of melanin.

JP2009-179605, titled “Melanogenesis inhibitor, and skin-lightening agent comprising the same”, published on 13 Aug. 2009, discloses a skin-lightening agent comprising an effective ingredient selected from the group consisting of pollen, bee pollen and extracts thereof. The disclosed agent is capable of exhibiting melanogenesis-inhibiting actions.

In KR20070109426, titled “A cosmetic composition containing an extract of typhae pollen”, published on 15 Nov. 2007, a composition comprising an extract of Typhae pollen is provided. The composition is described to inhibit the activity of tyrosinase and melanogenesis. It is also described to have anti-oxidative, skin wrinkle improving and irritation alleviating effects.

It is known that pollen contains nutritional compounds like carbohydrates, proteins, amino acids, lipids, vitamins, minerals and traces of micronutrients. In addition, pollen also contains significant amount of polyphenolic substances, mainly flavonoids.

Naturally occurring polyphenols derived from plants are known to have antioxidant properties. Recently, there has been an increase in the use of these polyphenolic compounds in cosmetics. Phenolics such as flavonols, isolated from plants, have also shown diphenolase inhibitory activity due to their free 3-hydroxyl group.

Despite the efficacy of the above compositions in producing whiter skin, alternatives are continuously being sought as there is a continuous global market demand for skin-whitening agents. More and more dark-skinned people desire lighter skin color. Most Asian women for instance, aspire for a fairer complexion.

It is therefore desirable to find a new source of plant-derived whitening agent for dealing with the conventional situation.

### SUMMARY OF THE INVENTION

The above and other problems are solved and an advance in the art is made by providing an external skin preparation comprising pollen extract of oil palm of the genus Elaeis guineensis for whitening skin. It is an advantage of an external skin preparation in accordance with this invention that the external skin preparation provides the use as a tyrosinase inhibitor agent in cosmetics, as well as in other useful applications. A second advantage of this invention is that the pollen extract of oil palm of the genus Elaeis guineensis exhibits a good radical scavenging activity and shows excellent melanogenesis-inhibiting actions, and thus is effective for whitening the skin.

In accordance with an embodiment of the invention, the external skin preparation comprises pollen extract of oil palm of the genus Elaeis guineensis in an amount between 0.05% and 1.00% by weight, based on the total weigh of the external skin preparation, for whitening skin.

In accordance with some embodiments of the invention, the pollen extract of oil palm of the genus Elaeis guineensis is an ethanolic fresh oil palm pollen extract. In accordance with other of these embodiments, the pollen extract of oil palm of the genus Elaeis guineensis is an ethanolic dried oil palm pollen extract, a methanolic fresh oil palm pollen extract or a methanolic dried oil palm pollen extract.

In accordance with some embodiments of the invention, the external skin preparation is topically applied to treat a skin condition selected from the group consisting of dark spots, freckles, age spots, dark circles under the eyes, hyperpigmentation, discoloration, and any combinations thereof.

In accordance with some embodiments of the invention, the external skin preparation is in a form of a cosmetic selected from the group consisting of a cream, a lotion, a foundation, an ointment, a gel, a foam and a sun screen.

### BRIEF DESCRIPTION OF THE DRAWINGS

The above and other advantages and features of this invention are described in the following detailed description and are shown in the following drawings:

FIG. 1 illustrating a chart showing the whitening effect of the pollen extract (in an amount of 0.25% w/v in 50% propylene glycol) in accordance with the present invention during daily application in volunteers for 12 weeks;

FIG. 2 illustrating a chart showing the melanin reducing index;

FIG. 3 illustrating a chart showing the relationship between skin humidity and the treatment group;

FIG. 4 illustrating a chart showing the relationship between skin fatigue resistance and the treatment group;

FIG. 5 illustrating a chart showing the relationship between skin firmness and the treatment group; and

FIG. 6 illustrating a chart showing the relationship between skin elasticity and the treatment group.

### DESCRIPTION OF THE INVENTION

The present invention has carried out extensive studies on pollen extract for a lengthy period of time, and consequently found that pollen extract of oil palm of the genus Elaeis guineensis exhibits a broad potential of application in cosmetic industry. As a result of continued studies on this finding, it was found that pollen extract of oil palm exhibits excellent melanogenesis-inhibiting actions. Based on this finding, the present invention was achieved.

The present invention provides an external skin preparation comprising pollen extract of oil palm of the genus Elaeis guineensis, in an amount effective for whitening of skin. Preferably, the external skin preparation comprises pollen extract of oil palm in an amount ranging from about 0.05% by weight to about 1.00% by weight, based on the total weight of the external skin preparation.

As used herein, the term “skin” refers preferably to the surface of the face and neck. However, the term “skin” can also include, without limitation, any other parts of a human body, including the hands, elbows, upper arm region, front area, back area, buttocks, thighs, knees, legs and feet.

The oil palm pollen extract used in the external skin preparation of the present invention was found to exhibit excellent melanagenesis-inhibiting actions, regardless of the physical properties of the external skin preparation when being applied for the external skin preparation. It was also found from clinical tests that the oil palm pollen extract has the effect of whitening skin, and is safe to use without causing any side effects on the skin.

The oil palm pollen extract used in the external skin preparation of the present invention is prepared from fresh pollen extract or dried pollen extract of oil palm of the genus Elaeis guineensis. The fresh or dried oil palm pollen extract may be mixed with ethanol or 50% propylene glycol in water to obtain ethanolic fresh oil palm pollen extract or ethanolic dried oil palm pollen extract respectively or they may be mixed with methanol to obtain methanolic fresh oil palm pollen extract or methanolic dried oil palm pollen extract respectively. Preferably, ethanolic fresh or dried oil palm pollen extract is used in the external skin preparation of the invention. More preferably, ethanolic dried oil palm pollen extract is used. Preferably, the ethanolic dried oil palm pollen extract is prepared by mixing and dissolving the dried oil palm pollen extract in 50% propylene glycol in water.

The external skin preparation of the present invention can be prepared using different bases. Examples of bases that can be used include cosmetics selected from the group consisting of a cream, a lotion, a foundation, a gel, a foam, a sun screen, etc. The bases may be in any suitable form including liquid, solid, cream and paste forms.

The external skin preparation of the present invention is topically applied to the skin. The frequency of topical application to the skin can vary widely, depending upon personal needs. The external skin preparation may be applied to skin having condition selected from the group consisting of dark spots, freckles, age spots, dark circles under the eyes, hyperpigmentation, discoloration, and any combinations thereof.

The following Examples further illustrate the present invention in detail but are not to be construed to limit the scope thereof.

### EXAMPLE 1

Principle: The oil palm pollen extract used in the present invention is evaluated with the irritation assay system to predict its potential to cause dermal irritation. The method used is based on the test sample application over a membrane of collagen, keratin and colorant which is in contact with a proteinous reagent. When irritating products come into contact with the membrane, the irritating products may alter or denature the membrane and provoke different levels of precipitation, according to the sample irritancy capacity. This precipitation is detected as an opacity that is evaluated by a spectrophotometer connected to an appropriate computer program to perform calculations and obtain results.

Protocol: Four doses, 50 μL, 75 μL, 100 μL and 125 μL, of each fresh and dried oil palm pollen extract (ethanolic and methanolic) and oil palm pollen extract in cosmetic base cream were tested. The assay was based on these two compounds:

(1) a semi-permeable membrane containing keratin, collagen and a colorant reactive, and

(2) a reactive solution; protein macromolecular matrix (globulins) and glycoproteins.

Different amounts of sample were placed in contact with the membrane which was submerged in the reagent solution at 25° C. for 24 hours, so that the product could pass through the membrane and come into contact with the protein matrix of the reagent solution. A standard curve was drawn with the four calibrated substances, whose dermal irritation potential was well known. The comparison between the optical density measurements of the sample and those obtained by the standards enabled the HIE (Human Irritancy Equivalent) calculation. From this figure, the irritancy of the sample was classified through the scale detailed in Table 1. The data analysis and the HIE were determined by a computer program. Two quality control solutions given with each kit were tested to ensure the correct assay performance. At least one of the results would correspond with the specifications described in the intern protocol. The computer program would calculate and inform whether the controls were correct or not, and whether the assay might or might not be considered valid, or qualified or not qualified, A maximum qualified score (MQS) of HIE was chosen as the point to predict dermal irritancy classification.

TABLE 1
Relationship of Human Irritancy Equivalent (HIE) score to irritancy
classification

| Human Irritancy Equivalent (HIE) score | Classificatton |
|---|---|
| 0.00-0.90 | Non-irritant |
| 0.90-1.20 | Non-irritant/irritant |
| 1.20-5.00 | Irritant |

Results: The dermal irritation assay results indicate that ethanolic and methanolic fresh oil palm pollen extract are classified as irritant with HIE score of 1.37 and 1.57 respectively. Meanwhile, ethanolic and methanolic dried oil palm pollen extracts are classified as irritant with HIE score of 1.23 and 2.02 respectively. However, ethanolic oil palm pollen extract (in an amount between 0.5% and 1.0% w/v) incorporated in cosmetic cream base formulation are classified as non irritant, with HIE score of 0.09 and 0.08 respectively.

TABLE 2
Predicted dermal irritancy classification for ethanolic fresh and dried
oil palm pollen extract

| | Ethanolic fresh | | Ethanolic dried | |
|---|---|---|---|---|
| | oil palm pollen | | oil palm | |
| | extract | | pollen extract | |
| Dose | HIE | | HIE | |
| μl | score | Classification | score | Classification |
| 50 | 0.93 | Non irritant/irritant | 0.81 | Non irritant |
| 75 | 0.93 | Non irritant/irritant | 1.06 | Non irritant/irritant |
| 100 | 1.18 | Non irritant/irritant | 1.12 | Non irritant/irritant |
| 125 | 1.37* | Irritant | 1.23* | Irritant |

TABLE 3
Predicted dermal irritancy classification for methanolic fresh and
dried oil palm pollen extract

| | Methanolic fresh | | Methanolic dried | |
|---|---|---|---|---|
| | oil palm pollen | | oil palm | |
| | extract | | pollen extract | |
| Dose | HIE | | HIE | |
| μl | score | Classification | score | Classification |
| 50 | 0.93 | Non irritant/irritant | 1.04 | Non irritant/irritant |
| 75 | 1.38 | Non irritant/irritant | 1.45 | Irritant |
| 100 | 1.29 | Non irritant/irritant | 1.68 | Irritant |
| 125 | 1.57* | Irritant | 2.02* | Irritant |

TABLE 4
Predicted dermal irritancy classification for ethanolic dried oil palm
pollen extract incorporated in cosmetic cream base formulation

| | |
|---|---|
| 0.5% extract in cream | 1.0% extract in cream |
| base formulation | base formulation |

| Dose | HIE | | HIE | |
|---|---|---|---|---|
| μl | score | Classification | score | Classification |
| 50 | 0.06 | Non irritant | 0.03 | Non irritant |
| 75 | 0.03 | Non irritant | 0.00 | Non irritant |
| 100 | 0.03 | Non irritant | 0.08* | Non irritant |
| 125 | 0.09* | Non irritant | 0.06 | Non irritant |

Notes:
*maximum qualified score

Example 2

In Vitro Anti-Tyrosinase Activity Evaluation

Principle: Anti-tyrosinase activity was measured using enzyme tyrosinase obtained from mushroom. The substrate of the enzyme is L-tyrosine and the reaction required the presence of the co-substrate, L-DOPA. The activity of tyrosinase was quantified following the detection of DOPAchrome at 475 nm.

Protocol: Evaluation of anti-tyrosinase activity was performed using ethanolic oil palm pollen extract.

Results: Percentage of tyrosinase inhibition is shown in Table 5.

TABLE 5

Tyrosinase inhibition (%)

| Extract | Methanol | Ethanol |
|---|---|---|
| Fresh oil palm | 23.4 ± 4.0 | 41.9 ± 5.50 |
| Dried oil palm | 60.0 ± 4.1 | 53.4 ± 12.7 |

### EXAMPLE 3

TABLE 6
Preparation of cream base preparation containing oil palm pollen extract

| | g/100 g |
|---|---|
| Water phase (A) | |
| Deionised water | Qs to 100 g |
| Glycerin | 1.0-5.0 |
| Phenonip | 0.5-0.9 |
| Natrosol | 0.2-1.0 |
| Xanthan gum | 0.2-2.0 |
| Oil phase (B) | |
| Medium chain triglycerides | 1.0-10.0 |
| Eutenol G | 0.5-6.0 |
| Glucate SS | 1.0-5.0 |
| Glucamate SSE | 1.0-6.0 |
| Glycerol monostearate | 0.5-5.0 |
| Isopropyl myristate | 0.5-6.0 |
| Hydrenol MY | 0.5-3.0 |
| Fancol VB | 0.5-5.0 |
| Sodium lactate | 0.5-5.0 |
| Disodium EDTA | 0.05-0.1 |
| BHT | 0.02-0.1 |
| Oil palm pollen extract | 0.10-0.25 |

Principle: This cream can be used for daily application on the skin of the face or body to lighten skin colour.

Protocol: In this Example, ethanolic oil palm pollen extract was used. The ethanolic oil palm pollen extract was prepared by dissolving the dried oil palm pollen extract in 50% propylene glycol in water. Phase A contains water soluble components and phase B contains oil soluble components (see Table 6). Both phases were heated to 80° C. until dissolution. Phase B was transferred to phase A, and mixed hornogenously for 5 to 10 minutes at a speed of 10,000 rpm. The emulsion was then cooled to 45° C. before the ethanolic oil palm pollen extract was added to it.

TABLE 7
Preparation of gel base preparation containing oil palm pollen extract

| Water phase (A) | g/100 g |
|---|---|
| Deionised water at pH 5 | Qs to 100 g |
| Ultrez 20 | 0.2-1.5 |
| Phenonip | 0.5-0.9 |
| Glycerin | 1.0-5.0 |
| Triethanolamine | 0.1-1.5 |
| Oil palm pollen extract | 0.10-0.25 |

Principle: This gel can be used for daily application on the skin of the face or body to lighten skin colour.

Protocol: In this Example, ethanolic oil palm pollen extract was used. The ethanolic oil palm pollen extract was prepared by dissolving the dried oil palm pollen extract in 50% propylene glycol in water. Phase A contains water soluble components (see Table 7). Deionised solution at pH 5 and ultrez 20 were heated to 60° C. with stirring until the mixture was homogenous. Phenonip and glycerin were then added to the homogenous solution. The solution was then cooled to 45° C. before triethanolamine and the ethanolic oil palm pollen extract were added to it.

### EXAMPLE 4

In Vivo Evaluation of Oil Palm Pollen Extract in Volunteers

Principle: In the evaluation of efficacy studies, 20 healthy volunteers (10 males and 10 females, average age between 20-40 years old) with normal healthy skin participated for 3 months. They were divided into two groups of 10.

Skin melanin: The initial melanin values were taken from both the left and right upper arms of each subject using Mexameter MX16 before application of the test sample.

Protocol: Each subject in Group A applied a test sample containing 0.25% w/v of ethanolic dried oil palm pollen extract incorporated in cosmetic cream in one upper arm twice daily and each subject in Group B applied a control containing mainly the cosmetic cream without ethanolic dried oil palm pollen extract also in one upper arm twice daily. The melanin content of each application site was measured every fourth week and calculated as a percentage reduction in melanin content relative to the initial melanin value (% whitening).

Results: The reading on the measurement of skin melanin decreases as shown in Table 8. The results are expressed as means±standard error for n=3 replicates.

TABLE 8
Skin melanin decreases in the presence of pollen extract in
cosmetic cream base

| | Group A (Treatment) | Group B (control) |
|---|---|---|
| | (mean ± std error) | (mean ± std error) |
| R0 | 257.83 ± 25.40 | 285.37 ± 34.41 |
| R1 | 248.23 ± 24.26 | 288.80 ± 34.63 |
| R2 | 233.47 ± 22.38 | 292.40 ± 34.63 |
| R3 | 171.20 ± 19.28 | 292.80 ± 35.42 |

The sample data being normally distributed, Paired t-Test was used to determine the significant difference of the results. The value for readings R1, R2 and R3 were compared to the initial reading R0. The application of cream containing ethanolic dried oil palm pollen extract resulted in a significant whitening of the skin when compared to their respective controls. The results for Group A are very significant since the p value is less than 0.05 (0.00). On the other hand, the results for Group B are regarded as not significant as the p value is greater than 0.05 (0.213).

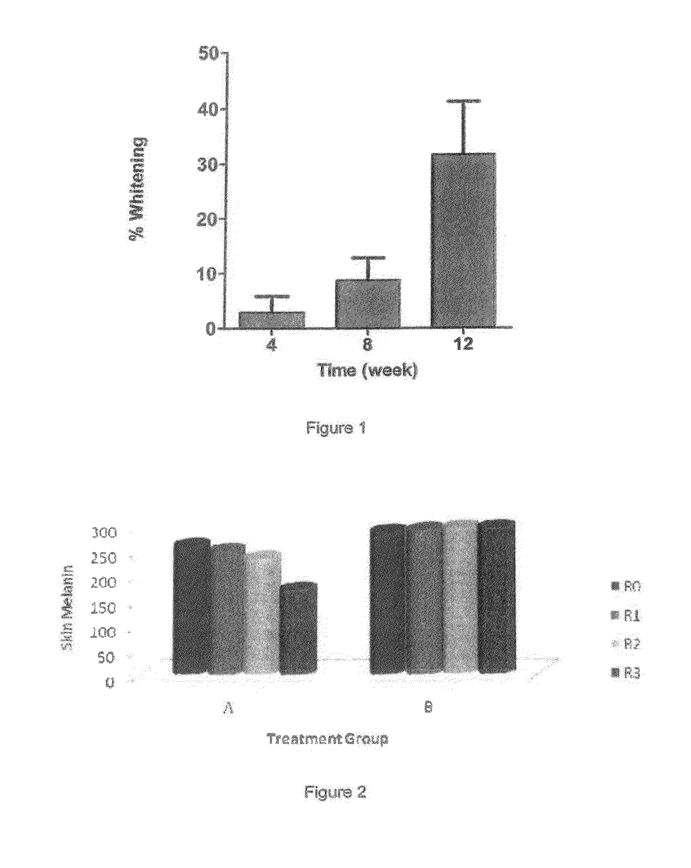

For each melanin value obtained, the percentage whitening for both groups was calculated as shown in FIG. 1, The application of the oil palm pollen extract resulted in significant whitening when compared to the control. The values of the percentage whitening increased gradually from week 4 through week 12. The volunteers treated with the preparation containing the oil palm pollen extract showed average maximum whitening at week 12, at 31.63%. This is a rapid increment as compared to weeks 8 and 4, which are at 8.78% and 2.87% respectively. Melanin reducing index is as shown in FIG. 2.

Skin hydration (humidity): Skin humidity is measured by a Corneometer 825 (Courage & Khazaka, Germany).

Protocol: Subjects were examined in a closed room with a controlled temperature and a relative humidity after remaining in the room for 30 minutes. Five measurements were performed on each testing area of different volar forearm points.

Results: The reading on the measurement of skin humidity increases as shown in Table 9. The results are expressed as means±standard error for n=3 replicates.

TABLE 9
Skin humidity increases in the presence of pollen extract in
cosmetic cream base

| | Group A (Treatment) | Group B (control) |
|---|---|---|
| | (mean ± std error) | (mean ± std error) |
| R0 | 43.16 ± 1.89 | 42.55 ± 1.73 |
| R1 | 54.43 ± 3.10 | 47.82 ± 3.75 |
| R2 | 61.37 ± 2.75 | 45.32 ± 2.56 |
| R3 | 73.77 ± 3.00 | 51.55 ± 3.27 |

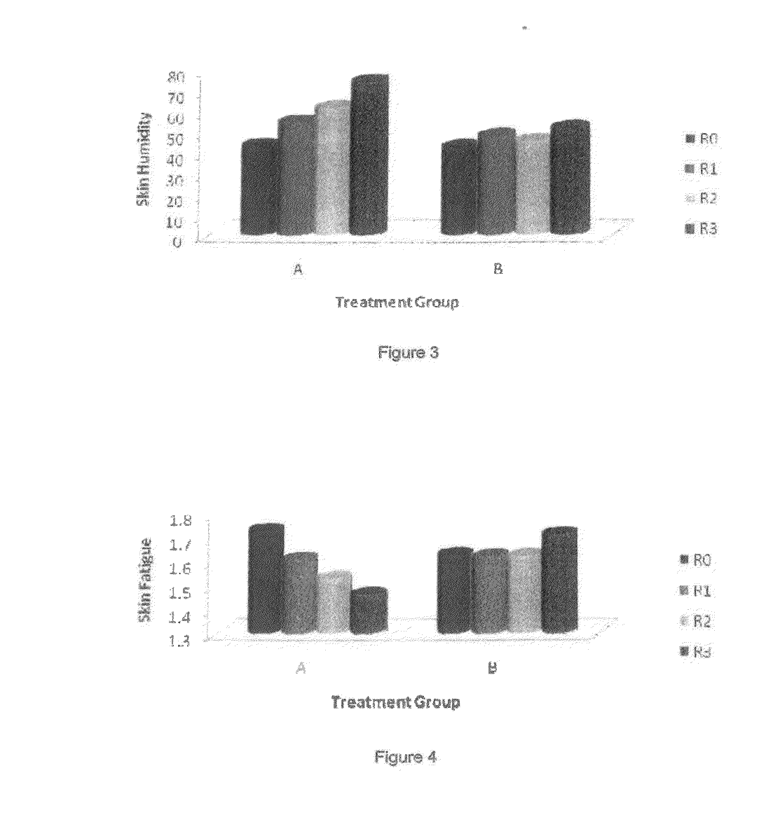

The data for skin humidity for Group A and Group B was presented as mean±standard error. Since the sample data is normally distributed, Paired t-Test was used to determine the significance of the result. The value for readings R1, R2 and R3 were compared to the initial reading R0. The analysis showed that the p value for Group A is equal to 0.000. Since the p value is less than 0.05, the result is regarded as very significant. On the other hand, the results for Group B are regarded as not significant as the p value is 0.050, which is not less than 0.05. The chart in FIG. 3 shows the relationship between skin humidity and the treatment group.

Biomechanical skin properties: The biomechanical skin properties that will be measured are elasticity, firmness and fatigue resistance, according to a method described in Muggli, R. (2005), Int. J. of Cosmetic Science, 27, 243-249. These skin parameters are assessed by a non-invasive suction- and elongation-method of a Cutometer MPA 580 (Courage & Khazaka, Germany).

Protocol: A 2-mm probe is used in time-strain mode (mode 1 with 10 repetitions) and a constant negative pressure of 450 mbar (4.5×10⁴ Pa) is applied for 4 seconds followed by a relaxation time of 2 seconds. Each measurement is performed in triplicate on each testing area of different volar forearm points (Yilmaz, E., Borchert, H. H., 2006, Int. J. of Cosmetic Science, 307, 232-238).

Results: The reading on the measurement of skin fatigue resistance decreases as shown in Table 10. The results are expressed as means ±standard error for n=3 replicates.

TABLE 10
Skin fatigue resistance decreases in the presence of pollen
extract in cosmetic cream base

| | Group A (Treatment) | Group B (control) |
|---|---|---|
| | (mean ± std error) | (mean ± std error) |
| R0 | 1.73 ± 0.12 | 1.63 ± 0.06 |
| R1 | 1.61 ± 0.10 | 1.62 ± 0.16 |
| R2 | 1.53 ± 0.10 | 1.62 ± 0.10 |
| R3 | 1.48 ± 0.06 | 1.71 ± 0.10 |

The data for skin fatigue resistance for Group A and Group B was presented as mean±standard error, Since the sample data is normally distributed. Paired t-Test was used to determine the significance of the result. The value for readings R1, R2 and R3 were compared to the initial reading R0. The analysis showed that the p value for Group A is 0.025. Since the p value is less than 0.05, the results are regarded as very significant. On the other hand, the results for Group B are regarded as not significant as the p value is 0.353, which is greater than 0.05. The chart in FIG. 4 shows the relationship between skin fatigue resistance and the treatment group.

Results: The reading on the measurement of skin firmness increases as shown in Table 11. The results are expressed as means±standard error for n=3 replicates.

TABLE 11
Skin firmness increases in the presence of pollen extract in
cosmetic cream base

| | Group A (Treatment) | Group B (control) |
|---|---|---|
| | (mean ± std error) | (mean ± std error) |
| R0 | 16.63 ± 1.20 | 20.60 ± 0.91 |
| R1 | 17.46 ± 0.89 | 15.80 ± 0.76 |
| R2 | 17.04 ± 0.89 | 14.68 ± 0.76 |
| R3 | 22.16 ± 1.36 | 18.49 ± 0.65 |

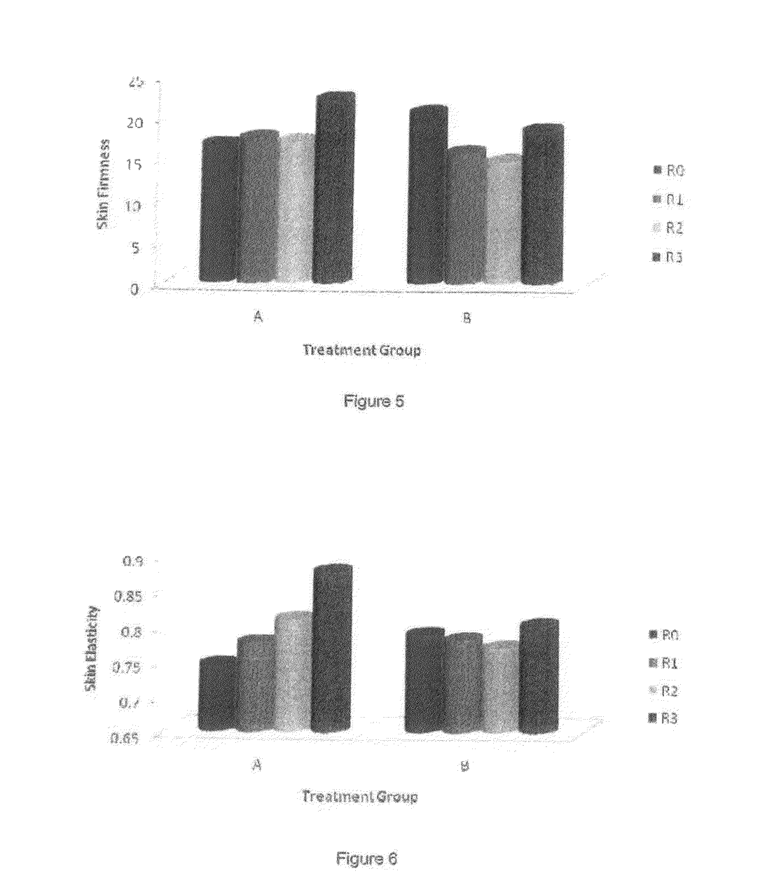

The data for skin firmness for Group A and Group B was presented as mean±standard error. Since the sample data is normally distributed, Paired t-Test was used to determine the significance of the result. The value for readings R1, R2 and R3 were compared to the initial reading R0. The analysis showed that the p value for Group A is 0.010. Since the p value is less than 0.05, the results are regarded as significant. On the other hand, the results for Group B are regarded as not significant as the p value for Group B is 0.063, which is greater than 0.05. The chart in FIG. 5 shows the relationship between skin firmness and the treatment group.

Results: The reading on the measurement of skin elasticity increases as shown in Table 12. The results are expressed as means±standard error for n=3 replicates.

TABLE 12
Skin elasticity increase in the presence of pollen extract in
cosmetic cream base

| | Group A (Treatment) | Group B (control) |
|---|---|---|
| | (mean ± std error) | (mean ± std error) |
| R0 | 0.75 ± 0.02 | 0.79 ± 0.01 |
| R1 | 0.78 ± 0.01 | 0.78 ± 0.01 |
| R2 | 0.81 ± 0.01 | 0.77 ± 0.02 |
| R3 | 0.88 ± 0.02 | 0.80 ± 0.01 |

The data for skin elasticity for Group A and Group B was presented as mean±standard error. Since the sample data is normally distributed, Paired t-Test was used to determine the significance of the result. The value for readings R1, R2 and R3 were compared to the initial reading R0. The analysis showed that the p value for Group A is 0.000. Since the p value is less than 0.05, the results are regarded as very significant. On the other hand, the results for Group B are regarded as not significant as the p value is 0.316, which is greater than 0.05. The chart in FIG. 6 shows the relationship between skin elasticity and the treatment group.

The above is a description of the subject matter the inventors regard as the invention and is believed that others can and will design alternative systems that include this invention based on the above disclosure.

1. An external skin preparation comprising pollen extract of oil palm of the genus Elaeis guineensis in an amount between 0.05% and 1.00% by weight, based on the total weight of the external skin preparation, for whitening skin.

2. The external skin preparation according to claim 1, wherein the pollen extract is a dried oil palm pollen extract dissolved in 50% propylene glycol in water.

3. The external skin preparation according to claim 1, wherein the pollen extract is a fresh oil palm pollen extract dissolved in 50% propylene glycol in water.

4. The external skin preparation according to claim 1, wherein the pollen extract is a fresh oil palm pollen extract dissolved in methanol.

5. The external skin preparation according to claim 1, wherein the pollen extract is a dried oil palm pollen extract dissolved in methanol.

6. The external skin preparation according to claim 1, wherein the preparation is topically applied to treat a skin condition selected from the group consisting of dark spots, freckles, age spots, dark circles under the eyes, hyperpigmentation, discoloration, and any combinations thereof.

7. The external skin preparation according to claim 1, wherein the preparation is in a form of a cosmetic selected from the group consisting of a cream, a lotion, a foundation, an ointment, a gel, a foam and a sun screen.

8. The external skin preparation according to claim 1, wherein the preparation is capable of inhibiting melanogenesis.

9. Use of a pollen extract of oil palm of the genus Elaeis guineensis for producing a skin whitening agent.

10. A whitening agent for skin comprising pollen extract of oil palm of the genus Elaeis guineensis in an amount between 0.05% and 1.00% by weight, based on the total weight of the whitening agent.

11. A cosmetic for skin whitening, comprising the external skin preparation according to claim 1.

12. A method of whitening skin comprising topically applying a preparation comprising extract of oil palm of the genus Elaeis guineensis in an amount between 0.05% and 1.00% by weight, based or the total weight of the preparation.
